(19) United States
(12) Patent Application Publication
(10) Pub. No.: US 2026/0069144 A1
(43) Pub. Date: Mar. 12, 2026

(54) INTRAOCULAR PRESSURE MEASURING DEVICE

(71) Applicant: MINGCHE BIOTECHNOLOGY CO., LTD, Suzhou (CN)

(72) Inventors: Kemin Wang, Suzhou (CN); Lijun Su, Suzhou (CN); Dongni Ren, Suzhou (CN); Chuanyan Xu, Suzhou (CN)

(21) Appl. No.: 19/387,686

(22) Filed: Nov. 13, 2025

(30) Foreign Application Priority Data
Apr. 23, 2025 (CN) 202510512349.1

(51) Int. Cl. A61B 3/16

(52) U.S. Cl. CPC A61B 3/165

(57) ABSTRACT

Provided is an intraocular pressure measuring device, consisting of an intraocular pressure sensor with a Fabry-Perot cavity, a shooting element consisting of a lens, and a shooting module consisting of a housing with a shooting hole, an optical path assembly in the housing, and a light source outside the housing. The light is transmitted to the Fabry-Perot cavity after passing through the optical path assembly, and forms an interference pattern. The shooting hole cooperates with the lens to obtain the interference pattern through the shooting element. With the shooting module adapted to existing shooting elements, the device can detect the intraocular pressure with an existing shooting element. The positions of the shooting module and the shooting element are adjustable according to the position of the Fabry-Perot cavity, so the incident light beam fulfills the normal incidence requirement, making it easier to adjust the angle during detection.

### CROSS-REFERENCE TO RELATED APPLICATIONS

The present claims the benefit of Chinese Patent Application No. 202510512349.1 filed on Apr. 23, 2025, the contents of which are incorporated herein by reference in their entirety.

### TECHNICAL FIELD

The present disclosure relates to the field of medical device technology, and particularly to an intraocular pressure measuring device.

### BACKGROUND

Glaucoma, as one of the three major causes of blindness in human eyes, is extremely harmful. High intraocular pressure is considered an important risk factor for the onset of glaucoma. Therefore, intraocular pressure is an important indicator in clinical practice to determine the treatment goals of glaucoma and to evaluate the treatment effect and prognosis.

At present, the main means of measuring intraocular pressure is to measure the patient's immediate intraocular pressure with instruments. The measurement instruments mainly include applanation tonometers and air-puff tonometers. Applanation tonometers have a complicated measurement process, with disadvantages such as requiring surface anesthesia before measurement, dripping sodium fluorescein on the cornea during measurement, and the measured value being affected by central corneal thickness. Compared with applanation tonometers, air-puff tonometers feature simpler processes of intraocular pressure measurement and do not require topical anesthesia or sodium fluorescein. However, air-puff tonometers also have problems, including patient eye discomfort caused by the impact of the airflow, high cost, and limited portability.

In addition, there are many studies on micro-implantable intraocular pressure sensors based on different principles. These studies share the following characteristics: (1) The sensor is separated from the detection device, and the sensing method is non-contact; (2) The sensor area and volume are small, ranging from hundreds of microns to a few millimeters; and (3) The sensor contacts the eye structure and is attached to the eyeball or implanted inside the eyeball.

According to the sensing principle, these implantable intraocular pressure sensors are mainly classified into three types, namely electrical sensing, microfluidic sensing, and optical sensing. Chen et al. designed a contact lens-based intraocular pressure sensor that is sensitive to pressure based on capacitance. The frequency of the LC oscillator formed by capacitance and inductance also changes with the pressure change, and the reading device is a large network analyzer. Agaoglu et al. used microfluidic chips to measure intraocular pressure, and implanted an artificial lens integrated with a microfluidic chip into the eyeball using cataract surgery technology. As the intraocular pressure fluctuated, the position of the liquid-gas interface of the artificial lens shifted, and the intraocular pressure value could be obtained by monitoring the position of the interface. For electric sensing, limited by the circuit structure and materials, it is difficult to reduce the sensor size to the sub-millimeter level, and the reading device is large and expensive. For microfluidic sensing, the stringent requirements for airtightness and the indirect sensing principle of photographic readings result in a bottleneck for miniaturization. For optical sensing, the volume of an optical sensor is generally smaller than that of an electrical sensor or a microfluidic sensor, so it has become the main research direction of implantable sensors.

Micro pressure sensors based on optical sensing in the prior art usually need to rely on a desktop microscope to capture optical interference patterns. During detection, only three-axis adjustment, namely x, y, and z axes, can be performed, but not three-dimensional angle adjustment. As a result, micro pressure sensors cannot meet the high dependence of light incident angle in optical interference pattern capture, which limits their application.

### SUMMARY

In order to solve the problem that the pressure sensor based on optical sensing in the prior art cannot perform three-dimensional angle adjustment during intraocular pressure detection, the present disclosure provides an intraocular pressure measuring device, which adopts a shooting module compatible with shooting elements in the prior art. During detection, the angle of the shooting element can be adjusted to make the light incident angle meet the requirements for capturing optical interference pattern.

The technical solution employed by the present disclosure to solve the technical problem is as follows:
an intraocular pressure measuring device, including an intraocular pressure sensor, a shooting module, and a shooting element, in which,
the intraocular pressure sensor is provided with a Fabry-Perot cavity;
the shooting module consists of a housing, an optical path assembly arranged in the housing, and a light source arranged outside the housing;
the housing is provided with a shooting hole;
the light emitted by the light source is transmitted to the Fabry-Perot cavity after passing through the optical path assembly, and forms an interference pattern;
the shooting element consists of a lens; and
the shooting hole is suitable for cooperating with the lens to capture the interference pattern through the shooting element.

Optionally, the optical path assembly includes a beam splitter cube and a plano-convex lens sequentially arranged in the shooting hole.

Optionally, the optical path assembly further includes a narrowband filter arranged between the light source and the beam splitter cube.

Optionally, the shooting element is a mobile phone, and a CMOS image sensor is provided in the mobile phone.

Optionally, the shooting module further includes a clamp, of which one end is connected to the shooting element, and the other end is connected to the housing.

Optionally, the intraocular pressure sensor includes a sensor body and a bracket connected to the sensor body.

Optionally, the bracket is of a plate-like structure.

Optionally, the bracket includes a mounting end and a fixing end connected to the mounting end.

Optionally, the fixing end is of an arc-shaped structure.

Optionally, the fixing end includes a wide section, a gradient section, and a narrow section connected sequentially, and the widths of the wide section, the gradient section, and the narrow section decrease in sequence.

Optionally, the bracket is provided with a drainage groove.

Optionally, the drainage groove includes a first groove-shaped structure longitudinally arranged along the bracket.

Optionally, the drainage groove further includes a second groove-shaped structure obliquely arranged on the wide section, and the second groove-shaped structure is connected to the first groove-shaped structure.

The present disclosure has the following beneficial effects:

With the shooting module adapted to shooting elements in the prior art, the intraocular pressure measuring device according to the present disclosure can detect the intraocular pressure with a shooting element in the prior art. Furthermore, the positions of the shooting module and the shooting element can be adjusted according to the position of the Fabry-Perot cavity in the intraocular pressure sensor, so the incident light beam fulfills the normal incidence requirement, significantly reducing the difficulty of angle adjustment during intraocular pressure detection, which makes the device more portable, efficient, and easy to use.

### BRIEF DESCRIPTION OF DRAWINGS

The present disclosure will be further described below with reference to figures and embodiments.

FIG. 1 is a schematic diagram of the structure of the intraocular pressure measuring device according to the present disclosure;

FIG. 2 is an exploded view of the intraocular pressure sensor according to the present disclosure;

FIG. 3 is an exploded view of the shooting module according to the present disclosure;

FIG. 4 is a schematic diagram of the structure of the optical path assembly according to the present disclosure; and

FIG. 5 is a schematic diagram of the effect of shooting angle on the integrity of optical interference pattern according to the present disclosure.

Numerical references: 1—intraocular pressure sensor; 11—sensor body; 12—bracket; 121—mounting end; 122—fixing end; 1221—wide section; 1222—gradient section; 1223—narrow section; 1224—anti-slip structure; 123—drainage groove; 1231—first groove structure; 1232—second groove structure; 2—shooting module; 21—housing; 211—shooting hole; 22—optical path assembly; 221—beam splitter cube; 222—plano—convex lens; 223—narrowband filter; 23—light source; 24—clamp; 241—C-shaped element; 2411—mounting groove; 2412—through hole; 242—threaded connector; 3—shooting element; 31—lens.

### DETAILED DESCRIPTION OF THE EMBODIMENTS

The present disclosure will now be described in further detail. The embodiments described below are exemplary and are intended to explain the present disclosure, but should not be understood as limiting the present disclosure. Based on the embodiments of the present disclosure, all other embodiments obtained by ordinary technicians in the art without creative work belong to the scope of protection of the present disclosure.

In order to make the above objectives, features, and advantages of the present disclosure more obvious and understandable, specific embodiments of the present disclosure will be described in detail with reference to the drawings below.

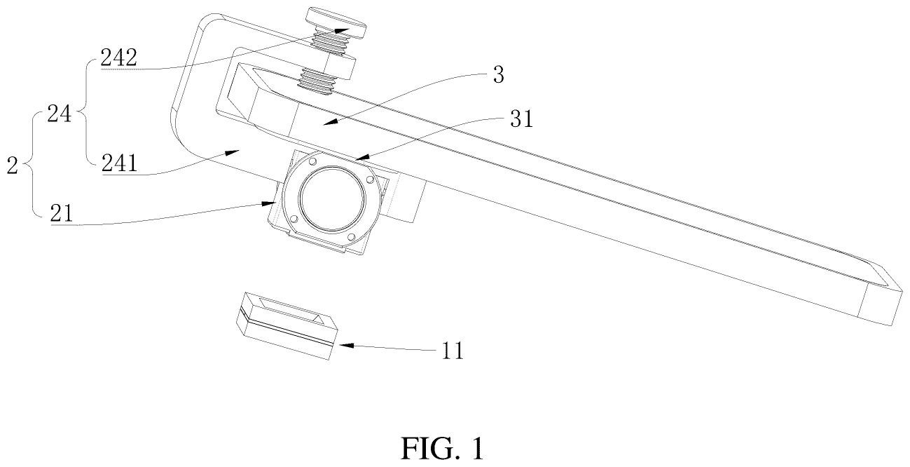

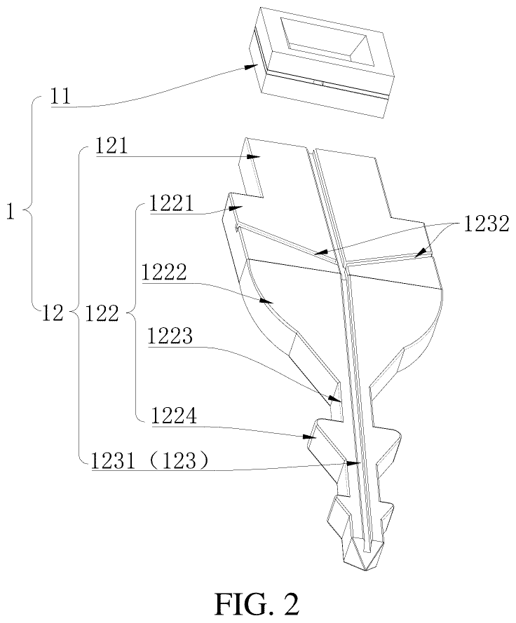

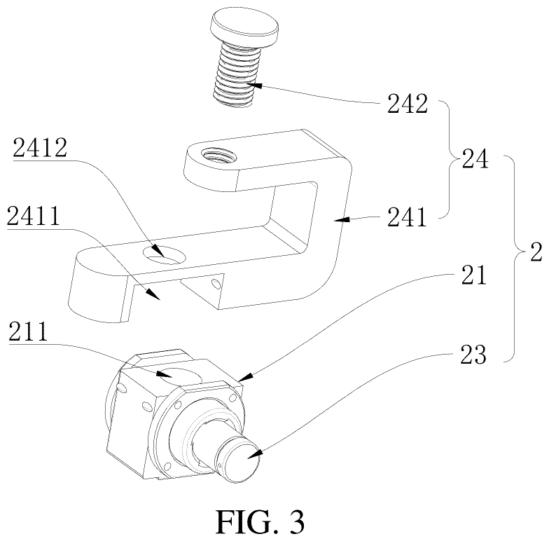

In order to solve the problem that the pressure sensor based on light sensing in the prior art cannot perform three-dimensional angle adjustment during intraocular pressure detection, the present disclosure provides an intraocular pressure measuring device as shown in FIG. 1, which includes an intraocular pressure sensor 1, a shooting module 2, and a shooting element 3. It should be noted that, in FIG. 1, the size of the intraocular pressure sensor has been artificially enlarged to clearly show the structure of the intraocular pressure measuring device. The intraocular pressure sensor 1 is a MEMS sensor, as shown in FIG. 2, and a Fabry-Perot cavity is provided on the intraocular pressure sensor 1. As shown in FIG. 3, the shooting module 2 includes a housing 21, preferably, the housing 21 is made of polylactic acid (PLA), and prepared with an extrusion 3D printing process. The optical path assembly 22 is arranged in the housing 21, and the light source 23 is arranged outside the housing 21. The housing 21 is provided with a shooting hole 211. The light emitted by the light source 23 is transmitted to the Fabry-Perot cavity after passing through the optical path assembly 22, and forms an interference pattern. The shooting element 3 includes a lens 31. The shooting hole 211 is suitable for cooperating with the lens 31 to obtain an interference pattern through the shooting element 3.

The light source 23 according to the present disclosure is preferably a white light source.

During operation, the light emitted by the light source 23 passes through the optical path assembly 22, and then normally comes into the Fabry-Perot cavity of the intraocular pressure sensor 1, which is an F-P resonator cavity, where the light is reflected by multiple surfaces, causing interferes to form an interference pattern. After passing through the optical path assembly 22, the obtained interference pattern is transmitted to the lens 31 of the shooting element 3, and the shooting element 3 takes pictures to capture the interference pattern in real time. Then, the real-time intraocular pressure can be obtained according to the interference pattern captured in real time.

Specifically, the method for obtaining the intraocular pressure according to the interference pattern can be selected from corresponding technologies in the prior art.

For example, a method is to implant the intraocular pressure sensor 1 in the anterior chamber of the eyeball, making the outer surface of the Fabry-Perot cavity contact the intraocular fluid to sense changes in intraocular pressure. When the intraocular pressure increases, the Fabry-Perot cavity deforms, causing the optical path of the reflected light to change, so that the generated interference pattern changes, and the interference fringes bend. By accurately identifying, segmenting, and extracting the interference fringe area of the interference pattern, the skeleton of the fringes after binarization is extracted, the order of each fringe is marked, and the central deflection of the film of the Fabry-Perot cavity after compression is calculated. Then, according to the one-to-one correspondence relation between the central deflection of the film of the Fabry-Perot cavity and the pressure on the film of the Fabry-Perot cavity calibrated by the calibration device, the photos containing interference fringe images can be converted into pressure values, and finally the intraocular pressure can be obtained according to the changes in the interference pattern.

The method for obtaining the intraocular pressure according to the changes in the interference pattern can also be selected from corresponding technologies in the prior art. For example, a method is to perform image processing and pressure solution on the interference pattern photographed by the shooting element 1 to obtain the intraocular pressure.

With the shooting module 2 adapted to shooting elements 3 in the prior art, the intraocular pressure measuring device according to the present disclosure can detect the intraocular pressure with a shooting element 3 in the prior art. Furthermore, the positions of the shooting module 2 and the shooting element 3 can be adjusted according to the position of the Fabry-Perot cavity in the intraocular pressure sensor 1, so the incident light beam fulfills the normal incidence requirement, significantly reducing the difficulty of angle adjustment during intraocular pressure detection, which makes the device more portable, efficient, and easy to use.

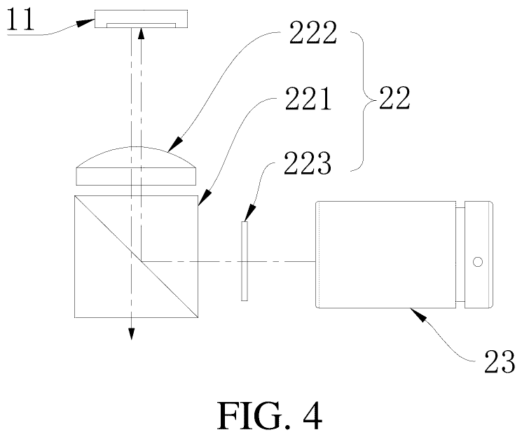

To achieve the measurement of intraocular pressure, as shown in FIG. 4, the optical path assembly 22 according to the present disclosure preferably includes a beam splitter cube 221 and a plano-convex lens 222 sequentially arranged in the shooting hole 211. The working wavelength range of the beam splitter cube 221 is from 450 nm to 650 nm. When the light comes in at an incident angle of 45°, the incident light can be divided into two beams of light at a ratio of about 50% transmission (T) and 50% reflection (R), with a tolerance of ±5% (T/R=50%: 50%±5%), so that the light emitted by the light source 23 is reflected by the beam splitter cube 221 to adjust the optical path direction, and then converged by the plano-convex lens 222 to the target plane for generating an optical interference pattern, where the Fabry-Perot cavity is located. The reflected light of the interference pattern is then transmitted to the lens 31 of the shooting element 3 after passing through the plano-convex lens 222 and the beam splitter cube 221, for collecting and imaging the interference pattern. The plano-convex lens 222 is preferably designed with a wavelength of 350 nm to 700 nm and a focal length of 20 mm.

Furthermore, the optical path assembly 22 according to the present disclosure further includes a narrowband filter 223 arranged between the light source 23 and the beam splitter cube 221, and the narrowband filter 223 is a 633 nm narrowband filter, so that the light emitted by the light source 23 is filtered by the narrowband filter 223 to obtain monochromatic light with a central wavelength of 633 nm and a bandwidth of ±10 nm.

The shooting element 3 according to the present disclosure can be any conventional digital camera, video camera, or smart phone including a CMOS image sensor. To further reduce the difficulty of shooting, a mobile phone is preferably used as the shooting element 3 according to the present disclosure, with a CMOS image sensor provided in the mobile phone to realize the acquisition and imaging of interference patterns.

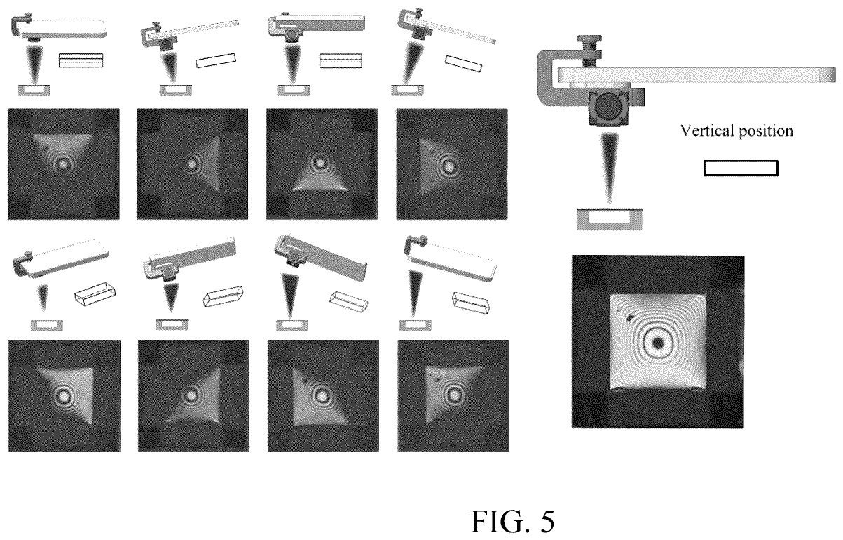

When an optical sensing-based pressure sensor in the prior art is used to measure the intraocular pressure, the incident light needs to fulfill the equal inclination interference, for which the main requirement is that the incident angle and the reflection angle (or the refraction angle) of the two interfering beams are equal when they are reflected or refracted. Specifically, the incident light beam needs to meet the requirement of normal incidence to ensure that the interference fringes are fully formed. When a micro pressure sensor is implanted in a pressure detection environment, it is impossible to guarantee that it is arranged horizontally due to its small size. When a desktop microscope is used, the microscope can generally only keep a vertical downward arrangement. When a complete optical interference pattern is expected, the only method is to adjust the spatial angle position of the object being measured by feel. This is extremely difficult, especially when the spatial angle position of the object being measured cannot be adjusted, for example, an intraocular pressure sensor implanted in the eye. Considering these issues, the present disclosure proposes an external shooting module 2 that can be adapted to any smart phone. Compared with adjusting the uncertain spatial angle position of the object to be measured, it is obviously much easier to adjust the angle of a mobile phone. Furthermore, the angle at which the mobile phone should be tilted can be determined according to the image captured in real time by its camera. The optical interference pattern captured in real time by the mobile phone camera is a square area. When the incident light is not normal to the interference plane, the square area is incomplete, appearing partially bright and partially dark. As shown in FIG. 5, the square interference area can be imagined as a sealed “square box” filled with water, and the bright part as a “bubble” in the sealed space. When the “bubble” is in a part of the square area, the “square box” will be tilted in the corresponding direction until the “bubble” is at the center of the square area. The mobile phone is exactly the “square box”. When the “bubble” is at the center of the square area, the incident light is in the normal direction to the interference plane. Then, the complete optical interference pattern is captured.

In order to facilitate connection with a mobile phone, the shooting module 2 according to the present disclosure preferably includes a clamp 24. One end of the clamp 24 is connected to the shooting element 3, namely the mobile phone, and the other end is connected to the housing 21.

The clamp 24 according to the present disclosure is preferably made of polylactic acid (PLA) and prepared with an extrusion 3D printing process. Further preferably, the clamp 24 is of a structure similar to a C-shaped clamp, including a C-shaped element 241 and a threaded connector 242. The C-shaped element 241 is connected to the housing 21. The C-shaped element 241 is connected to the shooting element 3 by a threaded connector 242. During use, the mobile phone is placed in the C-shaped element 241 and a connection is established by tightening the threaded connector 242. The opening range of the C-shaped element 241 according to the present disclosure is preferably 8 mm to 20 mm, which can be adapted to the thickness of most smart phones on the market.

Furthermore, the housing 21 and the clamp 24 according to the present disclosure are preferably connected in a buckle manner. Specifically, a mounting groove 2411 adapted to the housing 21 is preferably provided on the C-shaped element 241 of the clamp 24, a concave point is provided in the mounting groove 2411, and a convex point adapted to the concave point is provided on the outer side of the housing 21. In this way, the two parts can be easily assembled or disassembled by engagement or disengagement of the convex point and the concave point.

In addition, a through hole 2412 adapted to the shooting hole 211 is provided in the mounting groove 2411, so that the C-shaped element 241 will not affect light transmission.

In summary, the present disclosure provides an external shooting module 2 compatible with any smart phone. The housing 21 of the external shooting module 2 is made of environmentally friendly polylactic acid (PLA), and a precisely controlled extrusion 3D printing process is employed to ensure the consistency of structural strength and quality. The shooting module 2 integrates an optimized customized optical path design and high-performance optical elements, combined with real-time image capture, to achieve stable shooting of high-quality images. Compared with the first generation of desktop microscope image capture methods, this module not only ensures image clarity and optical image quality but also significantly reduces the complexity of user operation and minimizes the impact of jitter during shooting, making the device more portable and more efficient to operate, while improving its user experience and applicability.

The intraocular pressure sensor 1 according to the present disclosure can be any intraocular pressure sensor in the prior art that is provided with a Fabry-Perot cavity. Since the intraocular pressure monitoring device according to the present disclosure detects the intraocular pressure based on optical sensing, as mentioned above, during detection, the incident light needs to meet the requirement of equal-inclination interference, which requires that the incident light beam meet the requirement of normal incidence. Therefore, in order to ensure the clarity of the detection image, the intraocular pressure sensor 1 is required to be at a fixed position and should not move during detection. In order to avoid the intraocular pressure sensor 1 from moving during detection, the intraocular pressure sensor 1 according to the present disclosure preferably includes a sensor body 11 and a bracket 12 connected to the sensor body 11, so that the sensor body 11 can be fixed by the bracket 12, to reduce the difficulty of detection and improve the clarity of the detection image.

Brackets in the prior art used to fix intraocular implants are mostly cylindrical structures. For the intraocular pressure measuring device provided by the present disclosure, if the position of the sensor body 11 moves slightly during detection, the incident light beam will not normally come in, requiring readjustment of the shooting angle of the shooting element 3. Therefore, in order to ensure the stability of the position of the sensor body 11 during detection, the bracket 12 according to the present disclosure is preferably of a plate-like structure to increase the contact area between the bracket 12 and the inside of the eye and avoid the movement of the sensor body 11.

Specifically, the bracket 12 according to the present disclosure preferably includes a mounting end 121 and a fixing end 122 connected to the mounting end 121. The mounting end 121 is used to connect to the sensor body 11, and the sensor body 11 and the mounting end 121 according to the present disclosure can be connected by methods such as high-temperature bonding and compatible material bonding. The size of the mounting end 121 is determined according to the size of the sensor body 11.

Since an eyeball has a certain curvature, in order to improve the fit between the bracket 12 and the eyeball, thereby enhancing the stability of the sensor body 11 and the patient's comfort, the fixing end 122 according to the present disclosure is preferably of an arc-shaped structure, and the curvature of the arc-shaped structure is determined according to the curvature of the eyeball.

In order to improve comfort while ensuring the stability of the sensor body 11, the fixing end 122 according to the present disclosure preferably includes a wide section 1221, a gradient section 1222, and a narrow section 1223 connected sequentially, and the widths of the wide section 1221, the gradient section 1222, and the narrow section 1223 decrease in sequence.

It should be noted that for the bracket 12 according to the present disclosure, the direction in which the wide section 1221, the gradient section 1222, and the narrow section 1223 are distributed is the length direction, and the direction perpendicular to the length direction on the plane of the plate-like structure of the bracket 12 is the width direction.

Specifically, the wide section 1221 according to the present disclosure is preferably of a plate-like structure with a rectangular cross section to ensure the contact area between the bracket 12 and the eyeball. Preferably, the width of the gradient section 1222 decreases gradually, so that the width of the end connected to the wide section 1221 is the same as the width of the wide section 1221, and the width of the end connected to the narrow section 1223 is the same as the width of the narrow section 1223.

In order to take into account both comfort and the position stability of the sensor body 11, the length ratio of the wide section 1221, the gradient section 1222, and the narrow section 1223 according to the present disclosure is preferably (1.4 to 1.7):(1.1 to 1.4):(0.7 to 1).

In order to further improve the stability of the sensor body 11 after implantation, an anti-slip structure 1224 is preferably provided on the outer side of the narrow section 1223 according to the present disclosure, and specifically, the anti-slip structure 1224 is preferably of a convex structure extending outward along the narrow section 1223.

In order to take into account the stability and comfort of the position of the intraocular pressure sensor body 11, further preferably, at least two groups of protrusion structures are provided for the present disclosure, each group including two protrusions of the same size symmetrically arranged on the two sides of the narrow section 1223, and protrusion sizes gradually decrease in the direction away from the gradient section 1222.

Further preferably, the bracket 12 according to the present disclosure is provided with a drainage groove 123, so that the intraocular pressure measuring device according to the present disclosure has both the intraocular pressure measurement function and the drainage function to a certain extent.

Intraocular pressure sensors in the prior art usually only have the function of intraocular pressure detection, but not the drainage function. When the intraocular pressure is high, a corresponding drainage device is required to achieve the treatment effect. Considering this issue, the bracket 12 according to the present disclosure is preferably provided with a drainage groove 123, so that the aqueous humor can be diffused to the tissue around the eye through the drainage groove 123. In this way, the intraocular pressure sensor can have both intraocular pressure detection and drainage functions, so that intraocular pressure detection and drainage can be achieved through one implantation without increasing the number of implantations, surgically induced damage, or the patient's pain.

The drainage groove 123 includes a first groove structure 1231, which is a groove structure distributed along the longitudinal direction of the bracket 12 and sequentially penetrating the mounting end 121 and the fixing end 122. In order to further improve the drainage effect, the drainage groove 123 further includes a second groove structure 1232 obliquely distributed on the wide section 1221, and the second groove structure 1232 is connected to the first groove structure 1231.

The intraocular pressure sensor according to the present disclosure can be implanted into the eye by injection, significantly reducing the surgical trauma. With the intraocular pressure sensor according to the breakthrough design of the present disclosure that can be implanted into the eye by minimally invasive injection, the system can realize all-weather continuous monitoring of the intraocular pressure without electronic components or electromagnetic energy supply, and the intraocular pressure measurement accuracy reaches ±1 mmHg.

The intraocular pressure sensor according to the present disclosure can establish a functional relationship between the deflection of the Fabry-Perot cavity and intraocular pressure in the central area of the sensor, based on changes in the spacing of the sensor interference fringes caused by changes in the intraocular pressure. With the advanced M-net neural network training and sparse attention Transformer image inpainting algorithm integrated in the mobile phone APP, the intraocular pressure measuring device can automatically focus, identify, and clip the sensor interference fringe area and perform real-time intraocular pressure demodulation, so that patients can measure the intraocular pressure with mobile phones by themselves at home, and signal transmission does not rely on electromagnetic energy supply, effectively avoiding signal loss caused by external factors.

The intraocular pressure sensor according to the present disclosure has stronger compatibility and universality, as it can be compatible and used in conjunction with any ophthalmic implant device in the prior art, which is helpful to further explore the feasibility of new clinical technologies integrating glaucoma monitoring, diagnosis, and treatment.

Taking the above exemplary embodiments of the present disclosure as guidance, a person skilled in the art can make various changes and modifications based on the above description, without departing from the scope and spirit of the present disclosure. The technical scope of the present disclosure is not limited to the content disclosed in the specification, but must be determined in accordance with the scope of the claims.

What is claimed is:

1. An intraocular pressure measuring device, comprising an intraocular pressure sensor (1), a shooting module (2), and a shooting element (3), wherein
the intraocular pressure sensor (1) is provided with a Fabry-Perot cavity;
the shooting module (2) comprises a housing (21), an optical path assembly (22) arranged in the housing (21), and a light source (23) arranged outside the housing (21);
the housing (21) is provided with a shooting hole (211);
the light emitted by the light source (23) is transmitted to the Fabry-Perot cavity after passing through the optical path assembly (22), and forms an interference pattern;
the shooting element (3) comprises a lens (31);
the shooting hole (211) is suitable for cooperating with the lens (31) to obtain the interference pattern through the shooting element (3);
the optical path assembly (22) comprises a beam splitter cube (221) and a plano-convex lens (222) sequentially arranged in the shooting hole (211);
the optical path assembly (22) further comprises a narrowband filter (223) arranged between the light source (23) and the beam splitter cube (221);
the intraocular pressure sensor (1) comprises a sensor body (11) and a bracket (12) connected to the sensor body (11);
the bracket (12) comprises a mounting end (121) and a fixing end (122) connected to the mounting end (121);
the fixing end (122) comprises a wide section (1221), a gradient section (1222), and a narrow section (1223) connected sequentially, and the widths of the wide section (1221), the gradient section (1222) and the narrow section (1223) decrease in sequence;
the bracket (12) is provided with a drainage groove (123);
the drainage groove (123) comprises a first groove-shaped structure (1231) longitudinally arranged along the bracket (12); and
the drainage groove (123) further comprises a second groove-shaped structure (1232) obliquely arranged on the wide section (1221), and the second groove-shaped structure (1232) is connected to the first groove-shaped structure (1231).

2. The intraocular pressure measuring device according to claim 1, wherein the shooting element (3) is a mobile phone, and a CMOS image sensor is provided in the mobile phone.

3. The intraocular pressure measuring device according to claim 1, wherein the shooting module (2) further comprises a clamp (24), wherein one end of the clamp (24) is connected to the shooting element (3), and the other end of the clamp (24) is connected to the housing (21).

4. The intraocular pressure measuring device according to claim 1, wherein the bracket (12) is of a plate-like structure.

5. The intraocular pressure measuring device according to claim 1, wherein the fixing end (122) is of an arc-shaped structure.
